(19) United States
(12) Patent Application Publication
(10) Pub. No.: US 2019/0104795 A1
(43) Pub. Date: Apr. 11, 2019

(54) CONSTRUCTION HARDHAT WITH ENHANCED SAFETY FEATURES

(71) Applicants: Ivan Tirado, Sanford, FL (US); Peter Alexander Fabregas, Yankers, NY (US)

(72) Inventors: Ivan Tirado, Sanford, FL (US); Peter Alexander Fabregas, Yankers, NY (US)

(21) Appl. No.: 15/730,638

(22) Filed: Oct. 11, 2017

(51) Int. Cl. A42B 3/04; A42B 3/14; A61F 17/00

(52) U.S. Cl. CPC A42B 3/0406; A42B 3/0446; A42B 3/085; A42B 3/145; A61F 17/00; A42B 3/0453

(57) ABSTRACT

A safety hardhat consisting of a hard plastic body with a molded hump on top creating a cavity in the top interior of the body. An elastic retention system secures a soft pouch in the cavity created in the top interior forming a first aid kit and containing multiple first aid items. The safety hardhat can include a light source mounted on the rear of the plastic body providing a caution light, and/or a light source mounted on the front of the plastic body providing a spotlight. The light sources can include an LED.

### CROSS-REFERENCE TO RELATED APPLICATION(S)

Not applicable

### BACKGROUND OF THE INVENTION

### 1. Field of Invention

The present invention relates to the field of safety equipment, more particularly to hardhats worn by construction and other industrial workers.

### 2. Description of Related Art

A common article of safety equipment used almost universally by workers in construction and industry, the hardhat offers possible options for integrating additional safety equipment and enhance the utility of the hardhat. The hardhat can be enhanced with additionally integrated features making it more useful as a safety device or in an emergency. Based on the foregoing, there is a need in the art to enhance hardhats for increased safety and utility.

### SUMMARY OF THE INVENTION

In an embodiment, a safety hardhat, comprised of a hard plastic body with a molded hump on top creating a cavity in the top interior of the body, and an elastic retention system securing a soft pouch in the cavity functioning as a first aid kit and containing multiple first aid items.

In an embodiment, the safety hardhat further includes a holder attached to a side of the plastic body for holding a writing implement.

In an embodiment, the holder can be switched between the left side and the right side.

In an embodiment, the safety hardhat further includes a light source mounted on the rear of the plastic body providing a caution light.

In an embodiment, the safety hardhat further includes a light source mounted of the front of the plastic body providing a spotlight.

In an embodiment, the first aid items can be selected from the group consisting of band aids, antibiotic ointment, gauze pads, antibiotic cleaning pads, alcohol disinfecting pads, cotton swabs, latex gloves, tape (without a hard, rigid center), instant cold compress pouch, a tourniquet, an elastic bandage, and an inflatable splint.

In an embodiment, the caution light operates in at least the following modes—1) on, 2) off, and 3) flashing.

In an embodiment, the caution light comprises an LED.

In an embodiment, the spotlight comprises an LED.

In an embodiment, a safety hardhat comprises a hard plastic body with a molded hump on top creating a cavity in the top interior of the body. An elastic retention system secures a soft pouch in the cavity comprising a first aid kit and containing multiple first aid items. A light source mounted on the rear of the plastic body provides a caution light, and a light source mounted on the front of the plastic body provides a spotlight.

In an embodiment, the first aid items can be selected from the group consisting of band aids, antibiotic ointment, gauze pads, antibiotic cleaning pads, alcohol disinfecting pads, cotton swabs, latex gloves, tape (without a hard, rigid center), instant cold compress pouch, a tourniquet, an elastic bandage, and an inflatable splint.

In an embodiment, wherein the spotlight comprises an LED.

In an embodiment, wherein the caution light comprises an LED.

In an embodiment, the safety hardhat further comprises a holder attached to a side of the plastic body for holding a writing implement.

In an embodiment, the holder can be switched between the left side and the right side.

The foregoing, and other features and advantages of the invention, will be apparent from the following, more particular description of the preferred embodiments of the invention, the accompanying drawings, and the claims.

Advantages of the invention include:

1. Improved access to first aid equipment.

2. Improved consistent access through integrated storage.

3. Improved ease of use as the void between the head and hat is generally not used.

4. Improved chance of life saving potential.

5. Reduced need for additional containers or equipment dedicated to the first aid equipment carried by a worker.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a left side view of a hardhat incorporating improved safety options, according to an embodiment of the present invention;

FIG. 2 is a right side view of a hardhat incorporating improved safety options, according to an embodiment of the present invention;

FIG. 3 is a front/top view of a hardhat incorporating improved safety options, according to an embodiment of the present invention;

FIG. 4 is a back side view of a hardhat incorporating improved safety options, according to an embodiment of the present invention;

FIG. 5 is a bottom internal view of a hardhat incorporating improved safety options, according to an embodiment of the present invention; and

FIG. 6 is an expanded view of FIG. 5 showing the hardhat and the contents of the first aid kit safety enhancement, according to an embodiment of the present invention.

### DETAILED DESCRIPTION OF PREFERRED EMBODIMENTS

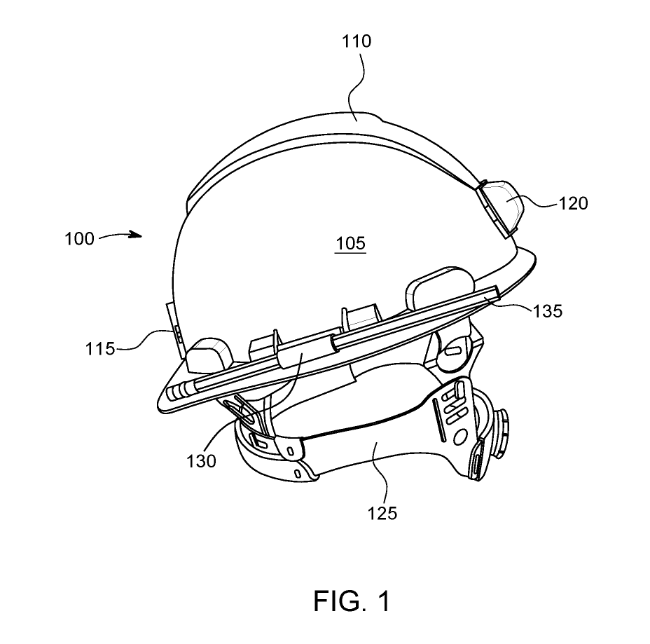

Preferred embodiments of the present invention and their advantages may be understood by referring to FIGS. 1-6, wherein like reference numerals refer to like elements.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The Hardhat Structure

FIG. 1 depicts a left side view of an exemplary embodiment of the safety hardhat of the disclosed invention. Hardhat 100 can consist of a hard plastic molded body 105. Incorporated in plastic body 105 is a molded hump 110 creating a cavity/compartment in the interior, an LED spotlight 115 on the front of body 105, a red LED caution light 120 on the rear of the body 105, an adjustable hardhat suspension system 125, pen/pencil/highlighter holder 130, and a pencil 135. The holder 130 can be used to hold a variety of different size writing implements. In an exemplary embodiment, the pen/pencil/highlighter holder 130 can be changed from the left side to the right side.

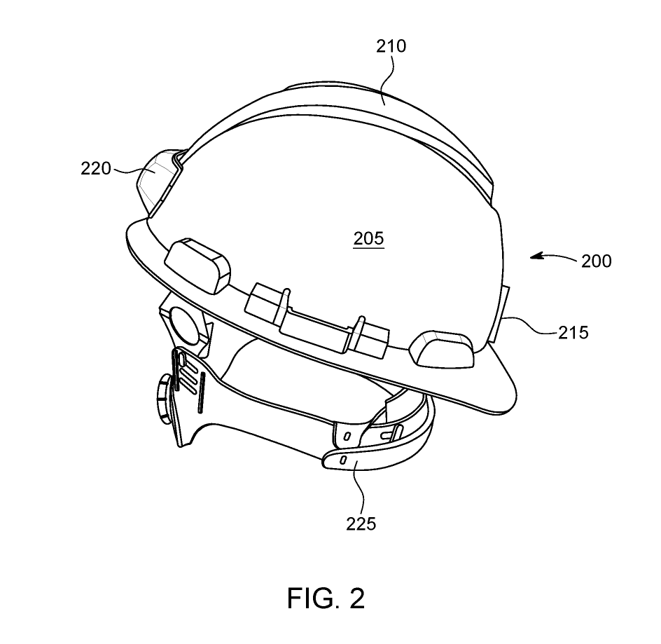

FIG. 2 depicts a right side view of an exemplary embodiment of the safety hardhat of the disclosed invention. Hardhat 200 can consist of a plastic molded body 205. The plastic body 205 can consist of a molded hump 210 creating a cavity/compartment in the interior, an LED spotlight 215 on the front of body 205, a red LED caution light 220 on the rear of the body 205, and an adjustable hardhat suspension system 225.

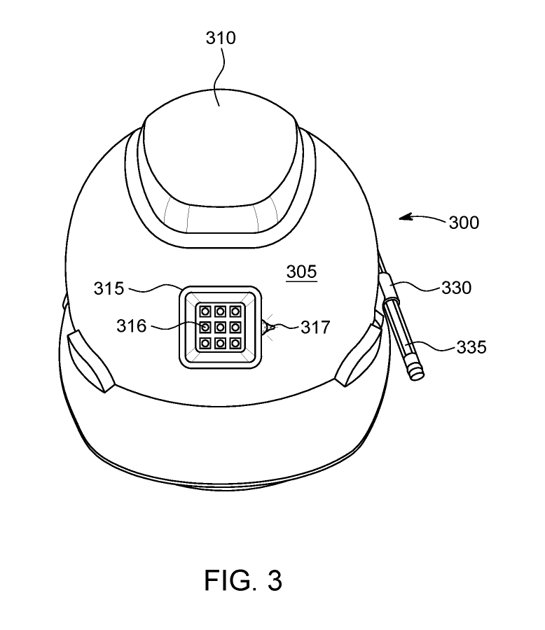

FIG. 3 depicts a front/top side view of an exemplary embodiment of the safety hardhat of the disclosed invention. Hardhat 300 can consist of a plastic molded body 305. The molded plastic body 305 can consist of a molded hump 310 creating a cavity/compartment in the interior, an LED spotlight 315 on the front of body 305, pen/pencil/highlighter holder 330, and a pencil 335. In an exemplary embodiment, LED spotlight 305 can include an LED light source 316 and on/off switch 317. The on/off switch 317 can include three positions: on; off; and flashing.

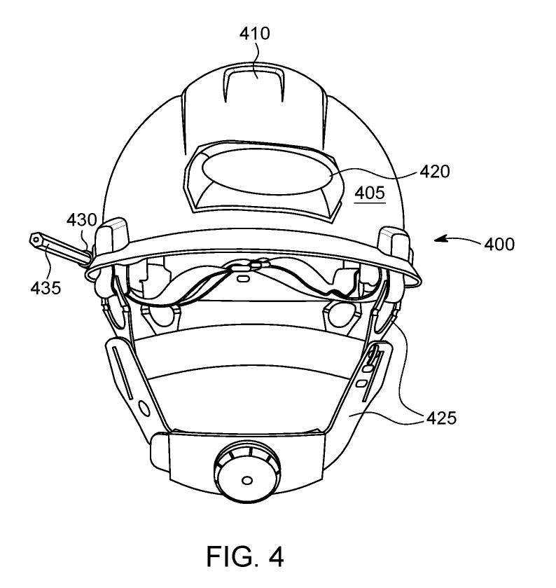

FIG. 4 depicts a rear view of an exemplary embodiment of the safety hardhat of the disclosed invention. Hardhat 400 can consist of a plastic molded body 405. The plastic body 405 can consist of a molded hump 410 creating a cavity/compartment in the interior, a red LED caution light 420 on the rear of the body 405, an adjustable hardhat suspension system 425, pen/pencil/highlighter holder 430, and a pencil 435. In an exemplary embodiment, LED caution light 420 can operate in three modes: off; on (generating a constant red light); and flashing.

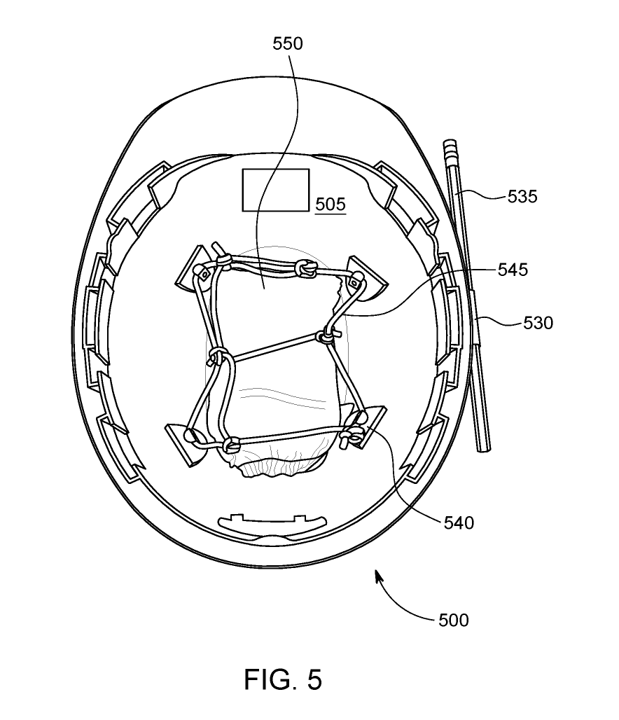

FIG. 5 depicts a bottom internal view of an exemplary embodiment of the safety hardhat of the disclosed invention. Hardhat 500 can consist of a plastic molded body 505, a pen/pencil/highlighter holder 530, and a pencil 535. A retention system inside the bottom interior can include a plurality of anchor points 540 anchoring an elastic retention cord 545 to the interior of the hardhat as shown. The retention system (as depicted four anchoring points 540 and elastic cord 545) can retain a first aid kit 550 in a cavity/compartment formed by humps 110, 210, 310, and 410. A suspension system (125, 225, and 435) as shown in FIGS. 1, 2, and 4 is not shown in FIG. 5. Elastic cord 545 can further comprise an elastic mesh net. The first aid kit 550 can consist of a soft, elastic pouch holding a variety of first aid items.

The First Aid Kit

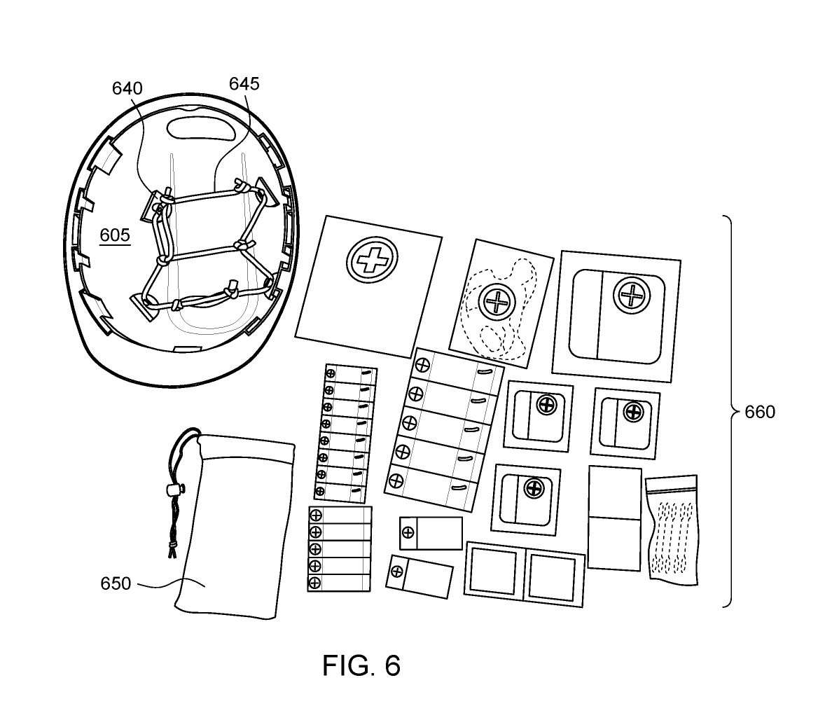

FIG. 6 is an expanded view of FIG. 5 showing the hardhat and an embodiment of the contents of the first aid kit 550. As shown, hardhat body 605 can include a retention system comprising a plurality of anchoring points 640 securing elastic cord 645 (i.e., an elastic mesh) in place to retain first aid kit 650 inside in a cavity/compartment formed by humps (110, 210, 310, and 410) in the bottom interior of hardhat body 605. First aid kit contents 660 can include band aids (multiple sizes and types), antibiotic ointment, gauze pads (several sizes), antibiotic cleaning pads, alcohol disinfecting pads, cotton swabs, latex gloves, tape (without a hard, rigid center), and an instant cold compress pouch. Other possible contents can include a tourniquet, inflatable splint, and an elastic bandage. All the materials in the first aid kit can be soft so as to not to harm the hardhat's wearer if an accident occurs.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A safety hardhat, comprising:
a hard plastic body with a molded hump on top creating a cavity in the top interior of the body; and
an elastic retention system securing a soft pouch in the cavity comprising a first aid kit and containing multiple first aid items.

2. The safety hardhat of claim 1, further comprising:
a holder attached to a side of the plastic body for holding a writing implement.

3. The safety hardhat of claim 2, wherein the holder can be switched between a left side and a right side.

4. The safety hardhat of claim 1, further comprising:
a light source mounted on the rear of the plastic body providing a caution light.

5. The safety hardhat of claim 1, further comprising:
a light source mounted of the front of the plastic body providing a spotlight.

6. The safety hardhat of claim 1, wherein the first aid items can be selected from the group consisting of:
band aids;
antibiotic ointment;
gauze pads;
antibiotic cleaning pads;
alcohol disinfecting pads;
cotton swabs;
latex gloves;
tape (without a hard, rigid center);
instant cold compress pouch;
a tourniquet;
an elastic bandage; and
an inflatable splint.

7. The safety hardhat of claim 4, wherein the caution light operates in at least the following modes—
1) on;
2) off; and
3) flashing.

8. The safety hardhat of claim 4, wherein the caution light comprises an LED.

9. The safety hardhat of claim 5, wherein the spotlight comprises an LED.

10. A safety hardhat, comprising:
a hard plastic body with a molded hump on top creating a cavity in the top interior of the body;
an elastic retention system securing a soft pouch in the cavity comprising a first aid kit and containing multiple first aid items;
a light source mounted on the rear of the plastic body providing a caution light; and
a light source mounted on the front of the plastic body providing a spotlight.

11. The safety hardhat of claim 1, wherein the first aid items can be selected from the group consisting of:
band aids;
antibiotic ointment;
gauze pads;
antibiotic cleaning pads;
alcohol disinfecting pads;
cotton swabs;
latex gloves;
tape (without a hard, rigid center);
instant cold compress pouch;
a tourniquet;
an elastic bandage; and
an inflatable splint.

12. The safety hardhat of claim 10, wherein the spotlight comprises an LED.

13. The safety hardhat of claim 10, wherein the caution light comprises an LED.

14. The safety hardhat of claim 10, further comprising:
a holder attached to a side of the plastic body for holding a writing implement.

15. The safety hardhat of claim 14, wherein the holder can be switched between a left side and a right side of the plastic body.
